(19) United States
(12) Patent Application Publication
(10) Pub. No.: US 2019/0038644 A1
(43) Pub. Date: Feb. 7, 2019

(54) PREPARATION FOR NASAL-NASOPHARYNGEAL TREATMENT

(71) Applicant: SUPRAPHARM CC, Rivonia (ZA)

(72) Inventors: Sheralee McDonald GREVES, Johannesburg (ZA); Wendy Sharon GENN, Johannesburg (ZA); Dimitri John TERPIZIS, Johannesburg (ZA); Costas Lambros TERPIZIS, Johannesburg (ZA)

(21) Appl. No.: 16/055,142

(22) Filed: Aug. 5, 2018

(30) Foreign Application Priority Data
Aug. 7, 2017 (ZA) ZA2017/05353

(51) Int. Cl. A61K 31/7004; A61K 31/047; A61K 47/10; A61K 47/18; A61K 9/00; A61P 11/02

(52) U.S. Cl. CPC A61K 31/7004; A61K 31/047; A61K 47/10; A61P 11/02; A61K 47/186; A61K 9/0043; A61K 9/0014; A61K 47/183

(57) ABSTRACT

Medicinal compositions or preparations or drugs for the treatment of disorders of the respiratory system such as the nasal-nasopharyngeal area. The preparation may include a carrier, between about 0.8% and 59% by weight/volume of a medicament selected from the group consisting of sugar monomers of the aldohexose series of carbohydrates; and a pharmaceutically effective amount of an antimicrobial preservative.

### FIELD OF THE INVENTION

THIS INVENTION relates to medicinal science and hygiene. More particularly, the invention relates to medicinal compositions or preparations or drugs for the treatment of disorders of the respiratory system.

### BACKGROUND TO THE INVENTION

In this specification, the following terms have the following meaning:
“Monomer” refers a molecule that can be bonded to other identical molecules to form a polymer.
“drug” refers to a chemical compound or composition with therapeutic activity.
“Sinusitis” refers to inflammation of the lining of the cavities in the bone surrounding the nose (the sinuses), usually as a result of a bacterial or viral infection spreading from the nose.
“Nasopharynx” refers to the passageway connecting the back of the nose to the top of the throat.
“Compound” refers to a substance composed of atoms or ions of two or more elements that are chemically combined or bonded together. Elements in a compound are present in definite proportions by mass and are bonded with each other in a specific manner and as such is a composition of multiple elements bonded together.
“Composition” refers to an aggregate material formed from two or more substances.
“Substance” refers to a species of matter of definite chemical composition.
“Sinuses” refers to a group of four paired air-filled spaces that surround the nasal cavity. The maxillary sinuses are located under the eyes; the frontal sinuses are above the eyes; the ethmoidal sinuses are between the eyes and the sphenoidal sinuses are behind the eyes.
“Cilia” refers to tiny, hair-like structures on the outside of some cells, providing mobility.
“Vasoconstrictor” refers to a medicament apt to cause narrowing of the muscular wall of blood vessels.
“Medicament” refers to a healing substance, composition, preparation, compound, medicine or remedy.
“Monosaccharide” refers to any of the class of sugars that cannot be hydrolysed to give a simpler sugar.
“Sugar” refers to any of the class of soluble, crystalline, carbohydrates found in living tissues.
“Carbohydrate” refers to a biomolecule consisting of carbon (C), hydrogen (H) and oxygen (O) atoms.
“Epimer” refers to each of two isomers with different configurations of atoms about one of several asymmetric carbon atoms present. The two isomers differ in configuration at only one stereogenic center.
“Stereoisomers” refers to isomeric molecules that have the same molecular formula and sequence of bonded atoms (constitution) but differ in the three-dimensional orientations of their atoms in space.
“Glucose” refers to a simple sugar with the molecular formula C₆H₁₂O₆, which means that it is a molecule that is made of six carbon atoms, twelve hydrogen atoms, and six oxygen atoms.
“hexoses” refers to monosaccharides with six carbon atoms, having the chemical formula C₆H₁₂O₆.
“aldohexoses” refers to a hexose having as a functional group, an aldehyde at position 1.
“aldehyde” refers to an organic compound containing the group —CHO, formed by the oxidation of alcohols.
“Mannitol” refers to a hexahydric sugar alcohol with the chemical formula being C₆H₁₄O₆.

Numerous preparations are known and used today in nasal-nasopharyngeal-treatment as many people suffer from nasal and sinus allergies and their complications such as bronchitis, colds, ear aches, sinusitis and sinus headaches. Most of these known preparations or medicaments attempt to cleanse or rinse the nasopharynx thereby to reduce mucous and bacteria residing therein.

In the early days, sea water was commonly used to rinse the nasal passage as part of a treatment for sinusitis. However, sea water by itself lacks effectiveness as substantial discomfort is associated with seawater rinsing.

As an alternative to sea water, nasal steroid sprays are available to treat allergies in the nasal passages. However, it is known that they have unwanted side-effects such as, for example, loss of smell, loss of activity of cilia, increased incidence of viral infection, nose bleeds, and the like. Further, several days or weeks may have to lapse before a benefit is obtained when steroids are used.

A vasoconstrictor, such as ephedrine, phenylephrine hydrochloride or oxymetazoline hydrochloride may alternatively be used as a decongestant, but these have various conceivable side-effects such as heart irregularities, increased blood pressure, and rebound phenomenon and use beyond three days is not generally advised.

The present invention, therefore, attempts to address, at least in part, the abovementioned shortcomings in known nasal passage treatments by providing a medicament, as set out in the summary and description of this patent specification, that can be used to effectively clean and clear the nose and sinus passages with a minimum of discomfort, and without the side effects common with existing nasal treatments.

### SUMMARY OF THE INVENTION

In broad terms, this invention provides a preparation for nasal-nasopharyngeal treatment, the preparation comprising: a carrier, between about 0.8% and 59% by weight/volume of a medicament selected from the group consisting of sugar monomers of the aldohexose series of carbohydrates; and a pharmaceutically effective amount of an antimicrobial preservative.

In an embodiment, the sugar monomer of the aldohexose series of carbohydrates is an isomer of glucose, preferably, selected from the group consisting of:

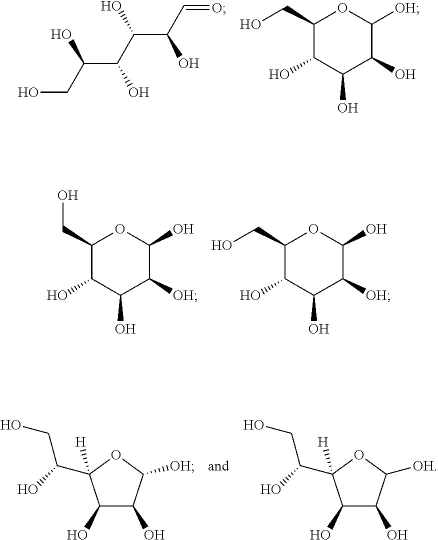

In an embodiment, the carrier is water such that the preparation is an aqueous solution, alternatively, the carrier may be an insufflatable substance that can be administered into a body cavity or a topically applied cream, foam, gel, lotion, ointment or balm.

In an embodiment, the antimicrobial preservative is selected from any one or more of the group consisting of: benzalkonlum chloride, phenylcarbinol, EDTA, benzyl alcohol, 2-phenylethanol, or 3-phenylpropanol, cetrimide, 3-cresol, chlorhexidine and organo mercurials.

The invention provides for the preparation to further optionally comprise between about 8% and 59% by weight/volume of

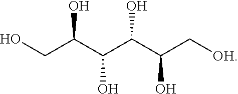 [SMILES: OC[C@@H](O)[C@@H](O)[C@H](O)[C@H](O)CO]

The preparation may comprise between about 0.45% and 0.95% (before buffering) by weight/volume of sodium chloride and the solution may be hypotonic.

In a preferred embodiment, the medicament comprises between about 0.8% and 59% by weight/volume D-mannose, alternatively, about 10% by weight/volume D-mannose, further alternatively, about 8% by weight/volume D-mannose.

The invention also extends to use of sugar monomers of the aldohexose series of carbohydrates in the manufacture of a medicament for treatment of a nasal-nasopharyngeal ailment in a patient, which treatment comprises administration of a pharmaceutically effective amount of said sugar monomer to said patient.

The invention further extends to a preparation for use in a method of treatment of a patient suffering from ailments selected from the group consisting of: nasopharyngeal congestion; hypersensitivity; inflammation; irritation associated upper respiratory infections; hay fever; post-nasal drip; sinusitis; and otitis media, which treatment comprises administration of a pharmaceutically effective amount of said sugar monomer to said patient.

Moreover, the invention extends to a process of manufacturing a preparation for nasal-nasopharyngeal treatment, the process comprising: providing a suitable carrier; admixing between about 0.8% and 59% by weight/volume of a medicament selected from the group consisting of sugar monomers of the aldohexose series of carbohydrates; and admixing a pharmaceutically effective amount of an antimicrobial preservative.

### BRIEF DESCRIPTION OF THE DRAWINGS

The invention is now described, by way of example, with reference to the accompanying non-limiting diagrammatic drawing, in which

FIG. 1 shows a stepwise flow diagram depicting a process of manufacturing a preparation for nasal-nasopharyngeal treatment, according to one embodiment of the invention.

### DETAILED DESCRIPTION OF THE INVENTION AND DRAWINGS

The invention relates to medicinal science and hygiene of the nasal and surrounding passages. More particularly, the invention involves the cleansing and rinsing of the nasopharynx to thereby reduce mucous and the bacteria residing therein.

The abovementioned minimization transposes into less problems with upper respiratory (specifically sinusitis) and minimization in post-nasal drip, resulting in easier breathing and less chance of developing chest infections.

The above may also apply to using D-mannose with or without saline and/or sorbitol for nasal irrigation or the use of a neti pot or in the form of a nasal gel, balm, ointment or spray. The spray may be used pre-sport, for allergies, runny blocked noses, before going to sleep, for nasal cleansing, and for general improvement of breathing through the nose.

Accordingly, this invention aims to provide a means to hygienize the nasopharynx and reduce the population of the pathogenic bacterial resident there.

In addition, this invention reduces infections of the nasopharynx and possible indications that may be associated with these infections.

Another objective of this invention is to play a role in clearing otitis, sinusitis and, making breathing easier in cases where asthma is triggered by inflammation of the upper respiratory tract.

This invention serves to efficiently deliver D-mannose/mannitol for the concurrent treatment of infections of the nasal passages, pharynx and upper respiratory infections.

To achieve the above the object is to develop a formulation, that is safe, versatile, efficient, stable and reliable, yet has a good storage life, is inexpensive plus easy to formulate and administer, and that respects the nasal mucosa.

The specific nature of the invention, as well as other objects, uses, and advantages thereof, will clearly appear from the following description.

Mucous membranes are the moist linings of the orifices and internal parts of the body that are in continuity with the external surface. They cover, protect, and provide secretory and absorptive functions in the channels and extended pockets of the outside world that are incorporated in the body.

This applies to:
the whole of the alimentary tract from the mouth to the anus;
the respiratory tract from the nose through the larynx, trachea, and bronchial tree leading to the microscopic millions of blind ends at the lung alveoli;
the urogenital tract—vulva, vagina, uterus, and Fallopian tubes in the female, urethra and bladder in both sexes reaching to the kidneys via the ureters, and the vas deferens and tubules reaching into each testis in the male.

The linings of all of these are epithelia and most are known as mucous membranes.

Since D-Mannose has mainly been used for UTI's, the dosage recommended for this treatment (500 mg every three to four hours for five days) was used as a guidance to determine the optimal concentration required for the nasal spray.

The nasal mucosa has a surface area of 96 square centimeters. The urinary tract (bladder) has a surface area ranging from 260-350 square centimeters.

This means that a total of 4 g of D-Mannose is administered daily for five days. After calculations it constitutes to 0.0069 mg/sq cm.

If this dosage is used to calculate the amount of D-Mannose per spray (0.3 ml) the amount of D-Mannose is 2.4 mg per spray which equates to 0.8% dosage strength. Then 0.8% spray would equate to 24 mg per 0.3 ml spray.

The nasal mucosa is at a PH of 6.3. Thus, for optimal nasal hygiene it is important for the spray etc., to be as close to 6.3 as possible, otherwise this would result in a burning sensation.

D-Mannose is a type of sugar found in a number of different fruits and vegetables, such as apples, peaches, blueberries, cranberries, cabbage, tomatoes, and green beans.

Dr. Hillary Martin at LiveStrong.com explains that D-mannose does not enter the blood stream, which means that it is not metabolized like other sugars and does not alter blood sugar levels.

D-mannose covers mucous membranes and surrounds bacterial cells, which makes it difficult, if not impossible, for infection-causing bacteria to stick to the walls of any mucosa.

Research has shown us a very important fact about D-Mannose. It is 10 times more active than the fructose in cranberries when it comes to inhibiting the adherence of E. Coli and other bacteria. If you can keep this form of bacteria from establishing itself, the majority of infections can be eliminated.

D-Mannose acts as an “antibacterial agent”. Bacteria have lectins on their surfaces that bind to the host cells, resulting in infection. However, D-Mannose competes with the bacterial lectins, occupying sites that would normally bind to the host cell D-Mannose receptors, thus preventing attachment and thwarting possible infection.

D-Mannose has proven to be particularly effective against Salmonella and E. coli. D-Mannose also has antiviral, anti-parasitic, and antifungal properties.

D-Mannose prompts anti-inflammatory activity and tissue regeneration. It appears to have an active role in the activation of macrophages, whose function it is to clean up debris which can cause inflammation. In fact, macrophages have at least four different receptors that bind D-Mannose implying that the sugar is vital for the proper function of these garbage collectors.

Further evidence for its anti-inflammatory abilities is based on studies involved in wound healing. D-Mannose stimulates fibroblasts to make more collagen and proteoglycans, which means that healing is speeded up, pain is lessened, and skin integrity returns to normal faster.

D-Mannose also works to correct over-active neutrophils (T cells) that cause misguided inflammation. Researchers in Australia have confirmed this action.

Evidently, D-Mannose displaces certain enzymes required for T Cells to flow into various areas such as the Joints, liver tissue, kidney's and even central nervous system areas, thereby acting as a natural anti-inflammatory agent. D-Mannose, Galactose, Fructose, and N-acetylneuraminic acid are all involved in reversing Failure to Thrive (FTT) syndromes. Failure to Thrive and cachexia (general ill health and malnutrition} are also seen in adults with AIDS and cancer.

It appears that a direct cause of FTT symptoms in children is the failure of glycoproteins to transport sugar, something that can actually start in the placenta. This malfunction of glucose transporters (glycoproteins) can also occur in the blood-brain barrier and muscle cells of the developing infant, inevitably producing a host of undesirable symptoms.

Research has shown that supplementation with glyconutrients resulted in significantly improved conditions. Apparently, it takes the combination of essential sugars to correct the problem and not just a supplementation of one sugar or of one nutrient.

Attachment of bacteria to mucosal surfaces of a susceptible host is now recognized as an important step in colonization and infection as per Jones, G. W. 1977. The attachment of bacteria to the surfaces of animal cells, p. 141-176. In J. L. Reissing (ed.), Microbial interactions. Chapman and Hall Ltd. London.

The ability of bacteria to adhere to epithelial cells in vitro appears to be mediated by the surface components of both cell types.

To probe the biochemical nature of the structure involved, investigators have focused on the sensitivity of bacterial agglutinins to carbohydrates which compete with identical or related structures on the cell surface as per Collier, W. A., and J. C. De Miranda. 1955. Bacterien-Haemagglutination. III. Die hummung der Coli-haemagglutination durch mannose. Antonie van Leeuwenhoek J. Microbial. Serol. 21:133-140.

Studies showing that D-mannose or its derivatives specifically inhibit adherence of Escherichia coli to epithelial cells support the hypothesis that E. coli ligans can recognise and bind D-mannose or D-mannose like receptors on the epithelial cell surface as per Eshdat, Y, I. Ofek, Y. Yashouv-Gan, N. Sharon, and D. Mirelman. 1978. Isolation of a mannose-specific lectin from Escherichia coli and its role in the adherence of the bacteria to epithelial cells. Biochem. Biophys. Res. Commun. 85:1551-1559.

Recent identification of D-mannose—insensitive strains of E. coli suggest that other bacterial ligans may also mediate attachment to epithelial cells as per Evans, D. G., and D. J. Evans, Jr. 1978. New surface E. COLI ADHERENCE TO UROEPITHELIAL CELLS 537 associated heat-labile colonization factor antigen (CFA/11) produced by enterotoxigenic Escherichia coli of serogroups 06 and 08. Infect. Immun. 21:638-647.

Investigations of E. coli strains isolated from urine suggest that several adherence factors are instrumental in the attachment of bacteria to human uroepithelial cells.

Svanborg Eden and Hansson (Svanborg Eden, C., and L. A. Hansson. 1978. Escherichia colipili as possible mediators of attachment to human urinary tract epithelial cells. Infect. Immun. 21:229-237) demonstrated D-mannose and amethyl-D-mannose—sensitive E. coli agglutination to guinea pig erythrocytes, but D-mannose insensitive adherence to uroepithelial cells.

In subsequent studies which were assessed adherence with radioisotopic rather than visual techniques, the addition of 2.5% D-mannose to E. coli uroepithelial cell suspensions completely inhibited adherence, even when the epithelial cells were highly receptive (22).

The purpose of this study was to further define D-mannose-sensitive adherence by investigating the effect of various carbohydrates on the adherence of E. coli to human uroepithelial cells. The results support the concept that adherence can be altered by specific carbohydrate molecules which compete with epithelial cell receptors for bacterial binding sites. This complex interaction is influenced by the ability of bacteria to adhere, the epithelial cell receptivity, and the concentration and time adherence to uroepithelial cells.

Specific carbohydrate molecules, which compete with epithelial cell receptors for bacterial binding sites.

Innate immunity is the earliest response to invading microbes and acts to contain infection in the first minutes to hours of challenge.

Unlike adaptive immunity that relies upon clonal expansion of cells that emerge days after antigenic challenge, the innate immune response is immediate.

Soluble mediators, including complement components and the mannose binding lectin (MBL) make an important contribution to innate immune protection and work along with epithelial barriers cellular defenses such as phagocytosis, and pattern-recognition receptors that trigger pro-inflammatory signalling cascades.

These four aspects of the innate immune system act in concert to protect from pathogen invasion.

The applicant's work has focused on understanding the protection provided by this complex defense system and, as discussed in this specification, the particular contribution of soluble mediators such as MBL and phagocytic cells.

Over the past two decades both human epidemiological data and mouse models have indicated that MBL plays a critical role in innate immune protection against a number of pathogens. As demonstrated by the applicants' recent in vitro work, it is shown that MBL and the innate immune signalling triggered by the canonical pattern-recognition receptors (PRRs), the Toll-like receptors (TLRs), are linked by their spatial localization to the phagosome.

These observations demonstrated a novel role for MBL, as a TLR co-receptor and establishes a new paradigm for the role of opsonins, which we propose to function not only to increase microbial uptake but also to spatially coordinate, amplify, and synchronize innate immune defenses mechanism.

In this review we discuss both the attributes of MBL that make it a unique soluble pattern recognition molecule and also highlight its broader role in coordinating innate immune activation.

The first level of response of the immune system is to try and wash out the irritated area. In upper respiratory infections this usually translates into nasal congestion because the immune system gets the fluid it needs for this washing and dilating blood vessel in the area.

The traditional response to these symptoms is to turn off the immune response by a decongestant or antihistamine. A treatment much more respectful of the wisdom of the immune system is to facilitate it in the attempt to wash the irritated area.

The applicant has discovered that direct contact of D-mannose/mannitol with saline to the nasal mucosa is the most effective way of producing the desired effects because there is no dilution of the actives and duration of contact is longer.

Preventing adherence of the bacteria to the nasal mucosa with the D-mannose and saline solution does not cause a problem with immune system with regard to resistance as the bacteria are simply washed out. Over and above this, the spray can be used more often and for longer periods of time as it does not create a rebound effect as seen with decongestant nasal sprays.

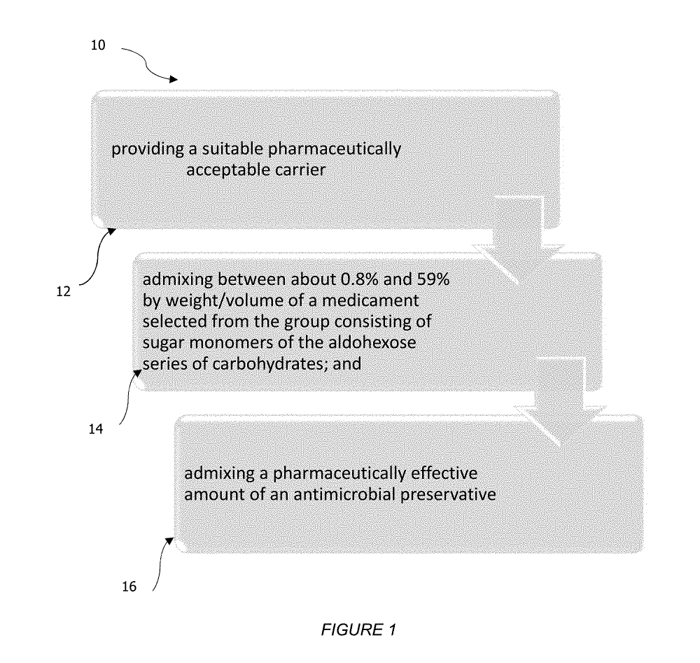

In use, a process of manufacturing (10) a preparation for nasal-nasopharyngeal treatment as shown in FIG. 1 of the Drawings, comprising firstly provision (12) for a suitable carrier. This is followed by admixing (14) between about 0.8% and 59% by weight/volume of a medicament selected from the group consisting of sugar monomers of the aldohexose series of carbohydrates (any stereoisomer of D-mannose, an epimer of Glucose). Finally, a pharmaceutically effective amount of an antimicrobial preservative is admixed (16) to the preparation.

The preparation may be, typically, a nasal spray formulated having approximately 8% D-Mannose/mannitol in an aqueous solution.

The spray is administered by a conventional spray bottle, or nasal irrigation using a neti pot or other nasal device.

As little as 0.8% D-mannose/mannitol in solution appears to be the effective minimum strength, the maximum strength is a saturated solution of 59 grams of D-mannose/mannitol per 100 millilitres of solution.

Mixing in a saline aqueous solution to facilitate one washing effect of the saline, the saline solution should be slightly hypotonic. The preferred saline solution is a 0.72% sodium chloride solution. (After buffering 0.65%)

Saline solution can be in the range from 0.40% sodium salt to 1% sodium salt. More than 1% sodium salt may result in a burning feeling of the mucus membranes in the nasal passages.

Sodium chloride is the preferred salt to make the saline solution although other compatible sodium compounds may be used, or the saline can be buffered using appropriate buffering agents.

The addition of sorbitol provides suitable glidant properties allowing the bacteria, allergens, pollutants and other germs to be easily swept away from the nasal cavity.

For infants administering two sprays of the solution about four to six times daily would suffice.

The D-Mannose preparation or solution could also be administered as drops from a dropper. If the solution were administered by drops, there would be approximately five (5) milligrams per drop, therefore, a recommended dosage by drops would be two drops in each nostril seven times a day. An excess amount is basically not harmful.

It is envisaged that the carrier for the D-Mannose preparation, in alternative embodiments of this invention, may be an insufflatable substance that can be administered into a body cavity or, further alternatively, even a topically applied cream, foam, gel, lotion, ointment or balm

Ultimately, this treatment is beneficial for nasal congestion. If used as described, results are evident, that a reduction of the population of resident pathogenic strep pneumonia and other bacteria with similar reduction in infections and inflammatory problems associated with these bacteria.

In conjunction with a first prescription of antibiotics, the use of D-Mannose, as described above, should usually be sufficient for treatment of most upper respiratory conditions where certain bacteria are the agents involved with the infection.

The essence of what has been shown and described above is only prototypical. We do not claim to have invented all the parts, elements or steps described.

Various variations can be made in the make-up, material, arrangement, and operation, and still be within the limits and scope of this invention.

The invention, for which patent protection is sought, is defined in the accompanying set of claims.

1. A preparation for nasal-nasopharyngeal treatment, the preparation comprising: a carrier, between about 0.8% and 59% by weight/volume of a medicament selected from the group consisting of sugar monomers of the aldohexose series of carbohydrates; and a pharmaceutically effective amount of an antimicrobial preservative.

2. The preparation of claim 1, wherein the sugar monomer of the aldohexose series of carbohydrates is an isomer of glucose.

3. The preparation of claim 1, wherein the isomer of glucose is selected from the group consisting of:

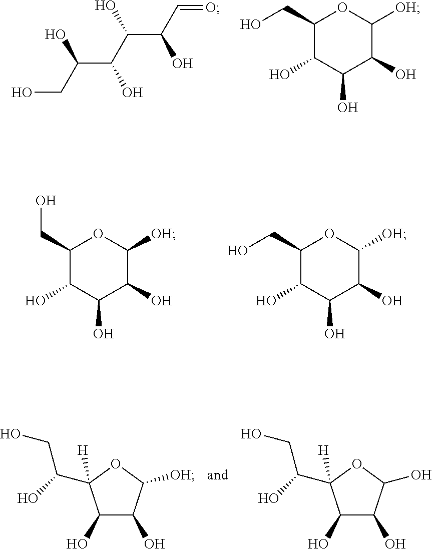 [SMILES: O=C[C@@H](O)[C@@H](O)[C@H](O)[C@H](O)CO ; OC[C@H]1OC(O)[C@@H](O)[C@@H](O)[C@@H]1O ; OC[C@H]1O[C@@H](O)[C@@H](O)[C@@H](O)[C@@H]1O ; OC[C@H]1O[C@H](O)[C@@H](O)[C@@H](O)[C@@H]1O ; OC[C@@H](O)[C@H]1O[C@H](O)[C@@H](O)[C@H]1O ; OC[C@@H](O)[C@H]1OC(O)[C@@H](O)[C@H]1O]

4. The preparation of claim 1, wherein the pharmaceutical carrier is water such that the preparation is an aqueous solution.

5. The preparation of claim 1, wherein the antimicrobial preservative is selected from any one or more of the group consisting of: benzalkonlum chloride, phenylcarbinol, EDTA, benzyl alcohol, 2-phenylethanol, or 3-phenylpropanol, cetrimide, 3-cresol, chlorhexidine and organo mercurials.

6. The preparation of claim 1, comprising between about 8% and 59% by weight/volume of

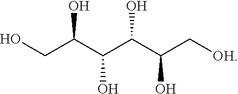 [SMILES: OC[C@@H](O)[C@@H](O)[C@H](O)[C@H](O)CO]

7. The preparation of claim 4, comprising between about 0.45% and 0.95% (before buffering) by weight/volume of sodium chloride.

8. The preparation of claim 4, wherein the solution is hypotonic.

9. The preparation of claim 1, wherein the medicament comprises between about 0.8% and 59% by weight/volume D-mannose.

10. The preparation of claim 1, wherein the medicament comprises about 10% by weight/volume D-mannose.

11. The preparation of claim 1, wherein the medicament comprises about 8% by weight/volume D-mannose.

12. The preparation of claim 1, in the form of a cream, foam, gel, lotion, ointment or balm that can be topically applied.

13. The preparation of claim 1, in the form of a substance that can be administered by insufflation into a body cavity.

14. A preparation as claimed in claim 1 for use in a method of treatment of a patient suffering from ailments selected from the group consisting of: nasopharyngeal congestion; hypersensitivity; inflammation; irritation associated upper respiratory infections; hay fever; post-nasal drip; sinusitis; and otitis media, which treatment comprises administration of a pharmaceutically effective amount of said sugar monomer to said patient.

15. A process of manufacturing a preparation for nasal-nasopharyngeal treatment, the process comprising: providing a suitable carrier; admixing between about 0.8% and 59% by weight/volume of a medicament selected from the group consisting of sugar monomers of the aldohexose series of carbohydrates; and admixing a pharmaceutically effective amount of an antimicrobial preservative.
